(19) United States
(12) Patent Application Publication
(10) Pub. No.: US 2012/0252107 A1
(43) Pub. Date: Oct. 4, 2012

(54) Bio-Reactor System and Method for Composting Food Waste

(76) Inventor: Tom Self, Tulsa, OK (US)

(21) Appl. No.: 13/437,620

(22) Filed: Apr. 2, 2012

Related U.S. Application Data
(60) Provisional application No. 61/470,320, filed on Mar. 31, 2011.

(51) Int. Cl. C12M 1/02; C12S 3/00

(52) U.S. Cl. 435/267; 435/290.2

(57) ABSTRACT

A bio-reactor made according to this invention uses low temperature aerobic composting to decompose bio-compostable material. The reactor includes mixing paddles with wiper blades which aerate and agitate a set of plastic resin biochips which house microorganisms and cause the chips to come into contact with bio-compostable material. A water pipe located toward the upper portion of the bio-reactor delivers fresh or recycled water (or some mix of the two) and the bio-reactor cycles between a water cycle and a non-water cycle. Agitation also cycles on and off. Perforated bottom screens limit the size of the composted material exiting the bio-reactor. The wiper blades, which may be brushes, continually wipe the bottom screens and work to prevent blockage and build-up of debris within the bio-reactor.

### CROSS REFERENCE TO PENDING APPLICATIONS

This application claims priority to U.S. Provisional Patent Application No. 61/470,320, filed Mar. 31, 2011.

### BACKGROUND OF THE INVENTION

Disposing of food waste and other bio-compostable materials typically occurs by collecting the waste at or near its point of generation and then hauling the waste to a landfill. Food waste that sits in an anaerobic state in a landfill produces methane gas. According to the Environmental Protection Agency, methane gas is significantly more harmful to the environment than CO₂. Also, landfill methods include oil- and gas-burning vehicles to haul and move the waste.

An alternate method is traditional composting. However, traditional composting tends to involve a rather lengthy process and, if the compost pile is not properly maintained, a foul odor may result. Additionally, composting is not available year-round in geographic locations which have below freezing temperatures. Further, traditional composting cannot be used on-site at certain locations, such as commercial food kitchens, restaurants and cruise ships, all of which generate large quantities of food waste.

An alternative to traditional composting is composting by means of a bio-reactor. Bio-reactors made by companies such as Bio-Ez/Waste-to-Water, Bio Hi-tech, and Green Key, are examples. The bio-reactor typically includes a tank into which food waste is introduced, agitated intermittently by means of paddles, and sprayed intermittently with fresh water. However, this type of bio-reactor uses a large amount of water. Keeping the micro-organisms alive in the tank of the bio-reactor is problematic, as is replenishing the organisms. Also, the entire composting process is extremely sensitive to having the right timing sequences among the water, agitation and rest cycles. Last, preventing unwanted discharge, while at the same time ensuring proper operation of the reactor, is difficult.

### SUMMARY OF THE INVENTION

Food waste and other bio-compostable material is introduced to a bio-reactor that has a high concentration of natural- and biologically produced organisms that assist the process in breaking organic material down. Along with these micro-organisms the bio-reactor introduces tap or fresh water at regular intervals and a motor turns several paddles inside the tank of the bio-reactor to create an aerobic environment for biodegradation to occur. Once the bio-compostable material is small enough to pass through a screen located in the base of the bio-reactor, the material is washed out as a manageable liquid effluent that can be reused or can be put into sewer line.

Any large food processing facility where there is an abundant quantity of organic food waste may make use of this invention. Examples are schools, hospitals, military facilities, corporate cafeterias, food processors or commissaries, supermarkets and farmers markets. On average 20-40% of their waste can be diverted from typical waste-disposal means and into a bio-reactor made according to this invention. The composting process continues 24 hours a day, 7 days a week with very limited interaction with the user other than introducing additional food waste and bio-compostable materials.

Objects of this invention are to provide a bio-reactor composting method and bio-reactor that (1) is more efficient, effective and reliable than current composting methods; (2) produces less odor than current bio-reactors, is quieter, and leaves no leftover sludge; (3) uses less water than current bio-reactors and produces no harmful liquids or gases; (4) is sleeker in design, requiring a smaller footprint and making controls and service connections more accessible; (5) provides a healthier sewer system without dangerous pathogens; (6) is an efficient, reliable, healthful waste management system; and (7) eliminates food-related cartage costs, minimizes excess waste management products, and eliminates risk of fines due to garbage overload

### BRIEF DESCRIPTION OF THE DRAWINGS

FIG. 1 is a partial cross-section isometric view of the interior drum of a food composting bio-reactor especially well-suited for practicing the composting method disclosed herein. The housing, which is preferably made of aluminum, contains a drum for receiving food waste to be composted. Within the drum is a plurality of black plastic media chips (“biochips”) that come into contact with the food waste and provide surface area for growing microorganisms useful in decomposing the food waste added to the interior of the housing. A water pipe, located in an upper portion of the drum, has nozzles that intermittently provide a misting of cold water. The cold water is preferably fresh water but could be re-circulated water or a combination of fresh and re-circulated water. A plurality of paddles driven by a motor agitates and aerates the food waste, biochips. microorganisms and water during the agitation cycle. The agitation cycle preferably is a continuous one, meaning the food waste is continuously agitated from the moment it is introduced into the drum until the moment it exits the drum as decomposed food waste. The decomposed food waste exits the housing by way of an outlet water stream. Bottom screens prevent the biochips and larger food waste particles from entering the outlet water stream.

FIG. 2 is an isometric view of the mixing paddle included in the food composting bio-reactor of FIG. 1. The paddles, which are preferably a one-piece construction with a v-shaped or angled front paddle end having an attached wiper blade, are secured to a rotating shaft, which is preferably made of stainless steel. The paddles are offset about 90° from one another. The paddles continue to turn throughout the composting method in order to keep the food waste in motion and the bottom screens clean, thereby preventing an overflow condition from occurring.

FIG. 3 is another view of the composting paddle, illustrating the connection of the wiper blades to the paddle.

FIG. 4 is view of the lower portion of the drum of the compositing bio-reactor of FIG. 1, illustrating the water pipes which are located beneath the bottom screens. One pipe with nozzles for flushing the bottom screen is located toward the front side of the bio-reactor and a second pipe with nozzles for flushing the screen is located toward the back side. A third pipe with nozzles for flushing the bottom pan is oriented transverse to the first two pipes and is located on the side of the bio-reactor opposite the discharge outlet side. The first and second pipes direct hot water upward toward the screens. The third pipe directs hot water toward the discharge outlet side. This third pipe may operate under a different timed cycle than the first two pipes.

FIG. 5 is a view of the inside of the drum of the bio-reactor of FIG. 1, illustrating the overflow sensor. An overflow sensor is located on each side of the housing.

FIG. 6 is a view of the bio-reactor of FIG. 1, illustrating the chain-and-gear arrangement used to drive the paddles. The upper gear is connected to the keyed shaft that drives the paddles

FIG. 7 is a view of an alternate embodiment of the chain drive tensioner of the bio-reactor of FIG. 6.

FIG. 8 is a partial cross-section isometric view of an alternate embodiment of the interior drum of a food composting bio-reactor especially well-suited for the composting method disclosed herein. Unlike the bio-reactor of FIG. 1, the bio-reactor of FIG. 8 does not include pipes for flushing the bottom screen, nor does it include a valve on the discharge water outlet to aid in the inoculation process

FIGS. 9A, B, C & D are front and side elevation views the housing of different sized bio-reactors of FIGS. 1 and 8.

### ELEMENTS USED IN THE DRAWINGS AND DETAILED DESCRIPTION

10 Bioreactor
20 Housing
21 Upper portion of 20
23 Lower portion of 20
25 Side of 20
27 Rear or back of 20
28 Access door
29 Water inlet
31 Hot water inlet
33 Water outlet
35 Power inputs
39 Trap or valve
40 Drum
41 Water pipe
43 End of 41
45 Spray nozzle
47 Paddle shaft
49 Key
50 Bottom screen
51 Perforations
53 Screen cleaner pipe
55 Nozzles
60 Base pan
61 Pan flush pipe
63 Nozzles
70 Mixing paddle
71 Paddle portion
73 Paddle end
74 Face surface
75 Wiper blade
77 Connecting rod portion
79 Keyway
85 Level sensors
90 AC Motor
91 Chain-drive and gear reducer
93 Chain tensioner
100 Biochips

### DETAILED DESCRIPTION OF THE PREFERRED EMBODIMENTS

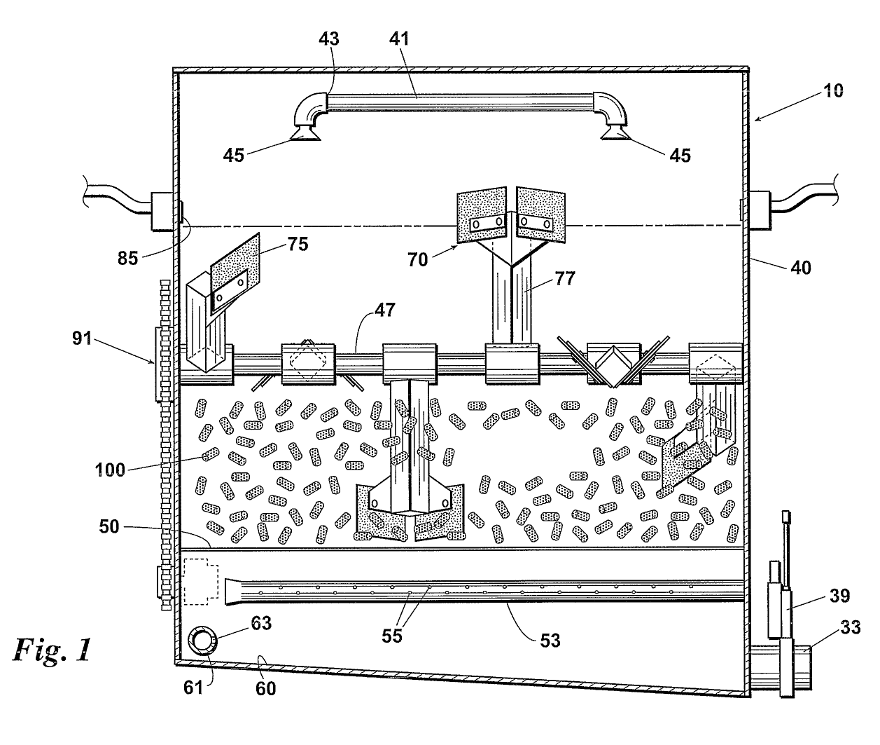

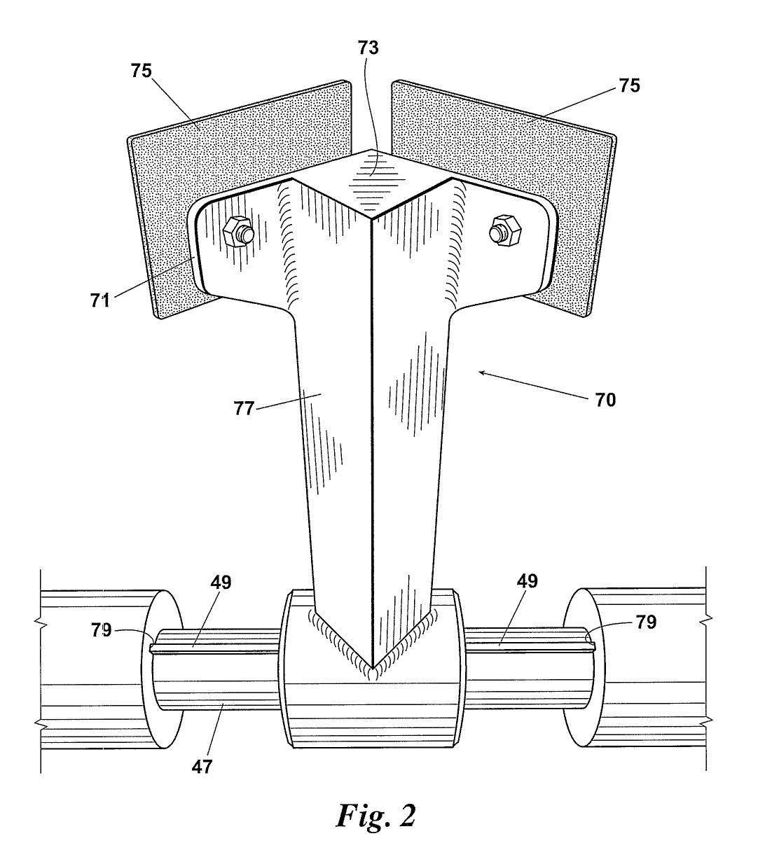

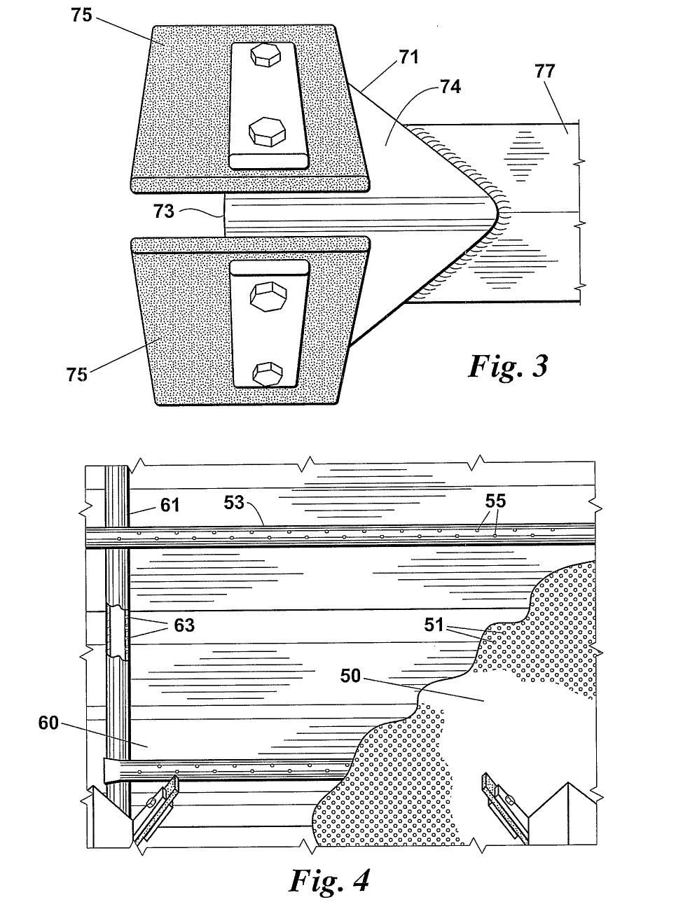

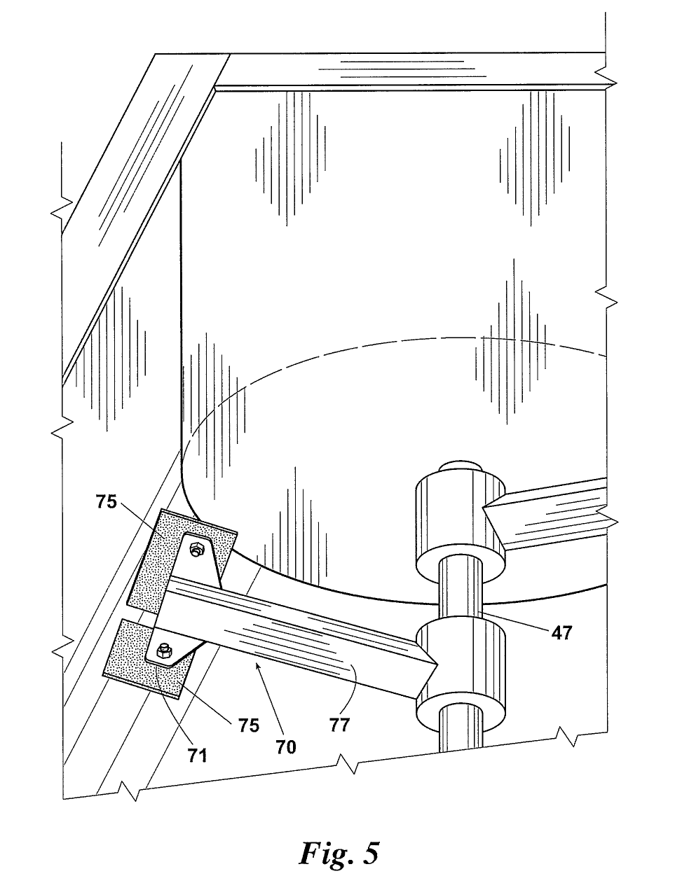

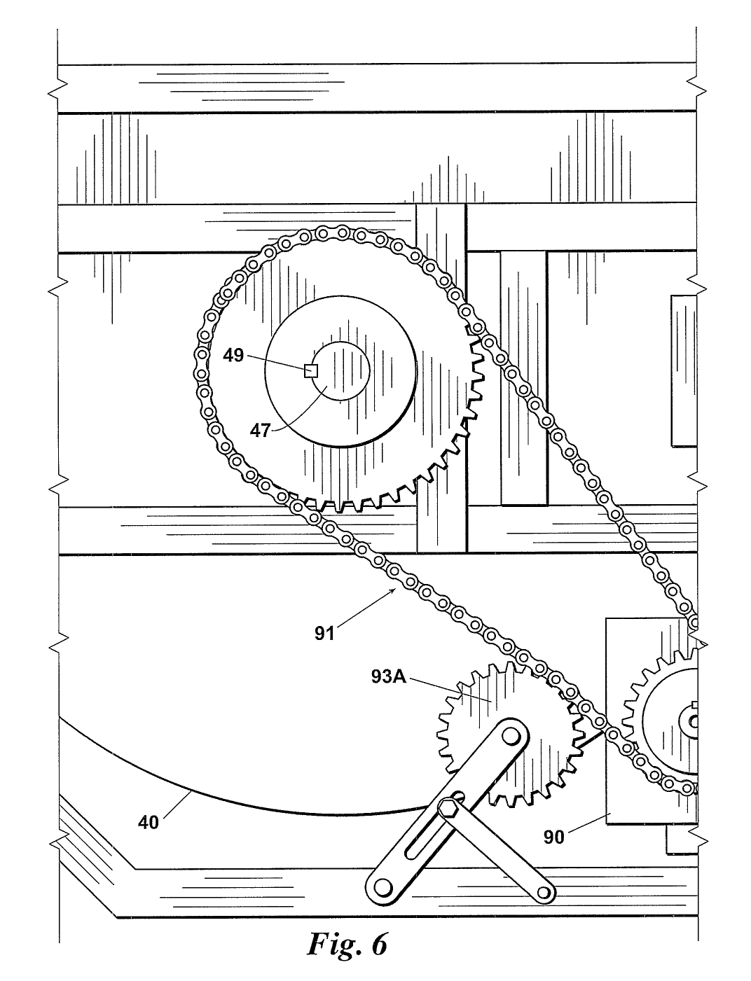

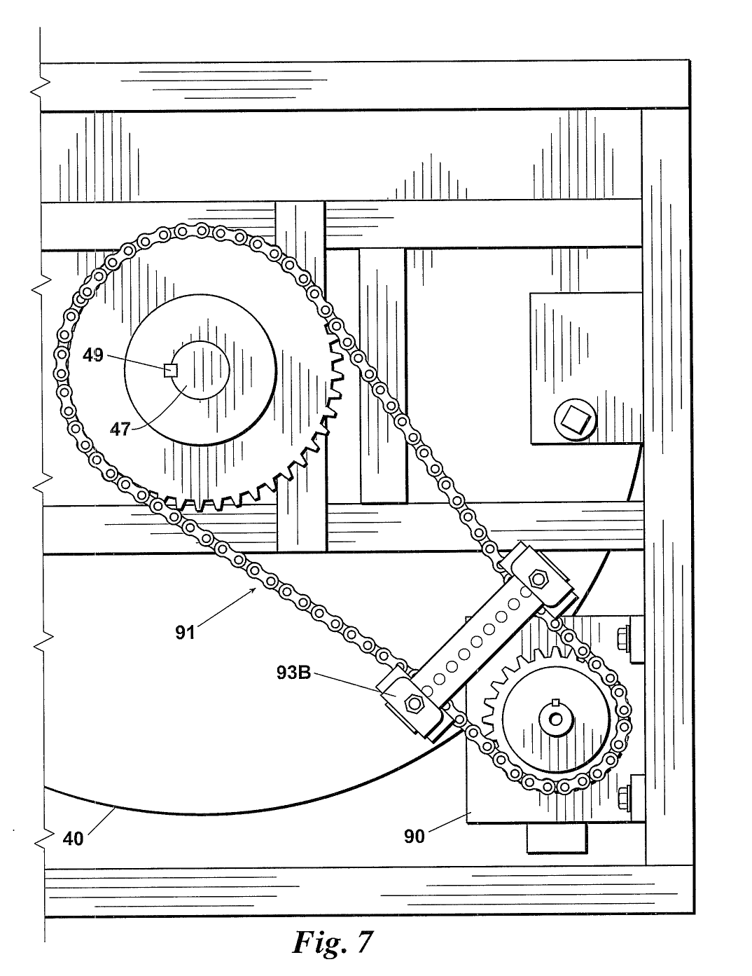

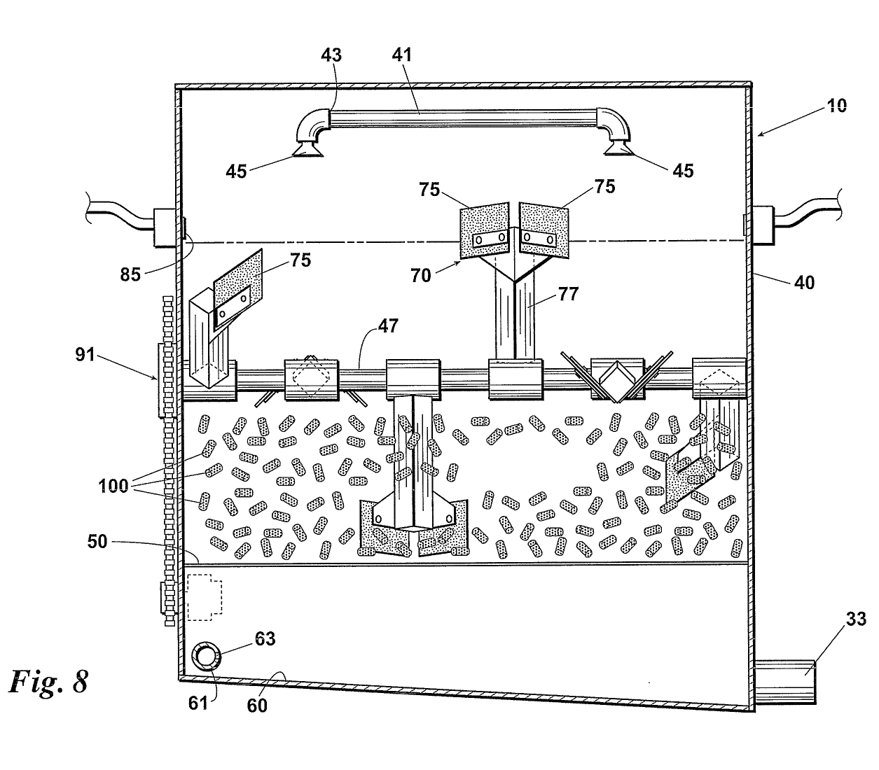

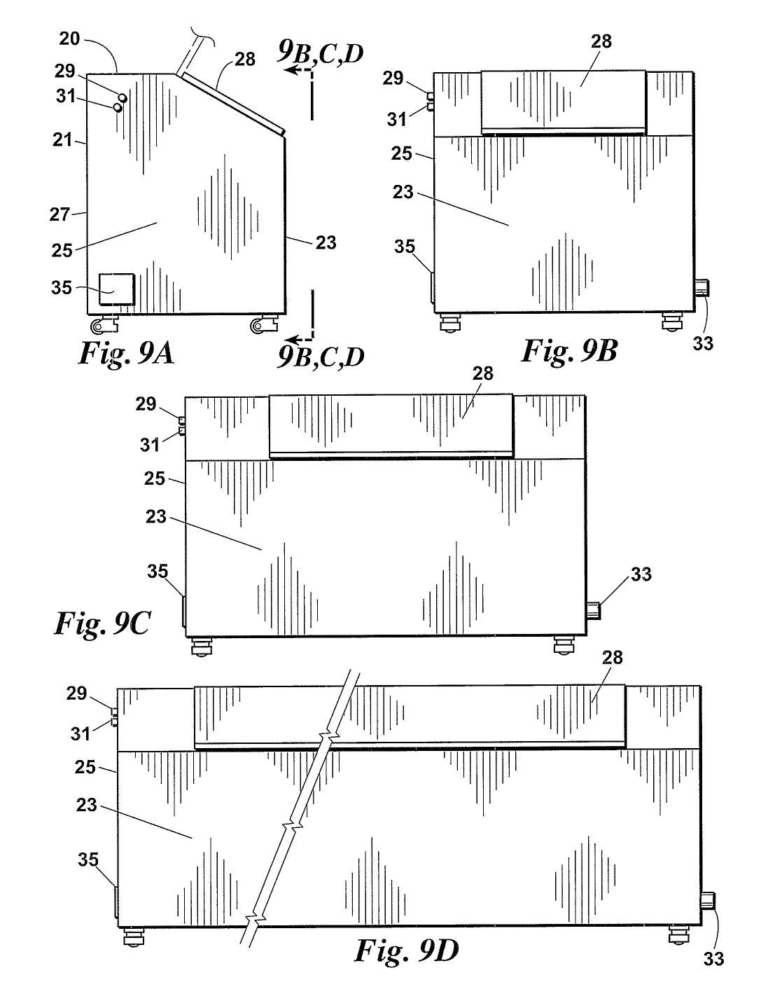

Referring to FIGS. 1-9, a method for composting food waste includes the steps of adding a food waste to a bio-reactor 10 and cycling the food waste between a water cycle, an agitation cycle, and a rest cycle. A bio-reactor 10 made according to this invention and practicing the method disclosed herein can be sized to process between 400 to 2,400 pounds of garbage every day and turn it into water. Further, the bio-reactor 10 can be located near or at the point where the waste is generated. Additionally, the bio-reactor 10 can dispose of bio-compostable materials, including but not limited to plates, cups, cutlery and straws in the same manner.

The bio-reactor 10 uses low temperature aerobic composting to control odor and contains a plurality of black plastic media chips (“biochips”) 100 that provide high surface area for harboring and growing micro-organisms useful in decomposing the food waste added to the drum 40 located in the interior of the housing 20 of the bio-reactor 10. The biochip 100 is a plastic resin-based material about the size of a small pellet. Each biochip 100 is slightly porous on its opposing ends. Preferably, the drum 40 of the bio-reactor 10 is filled with the quantity of biochips 100 necessary to bring the total level of biochips 100 to about 2 inches below the shaft 47 which drives the mixing paddles 70.

The microorganisms can be introduced automatically via a simple pump (not shown). To achieve the initial inoculation, a pneumatic trap or valve 39 located toward the lower portion 23 of the bio-reactor 10 remains closed to fill with the drum 40 with an appropriate amount of water. Milk and sugar are added to the micro-organism, biochips 100, and water mixture, and the mixture is continuously agitated for about 8 to 10 hours prior to introducing any food waste into the drum 40. In another embodiment of bio-reactor 10, the valve 39 is not used and inoculation occurs through a liquid bacteria sprayed from above. The micro-organisms decompose the food waste into a liquid effluent and trace amounts of CO₂. The effluent is then discharged through a water outlet 33. Depending on facility location and desired level of filtration, the possibilities for the effluent can range from irrigation, compost tea, non-potable plumbing, and potable water. The amount of effluent produced is approximately the weight in water of the food waste introduced to the bio-reactor 10.

The water cycle preferably includes a fresh water source. The amount of fresh water deployed during the water cycle may vary and the amount of time during which fresh water is deployed is based upon such factors as food-type, processing time, input frequency and total waste. Some preferred water cycles, arranged in order from a heavy duty cycle to a light duty cycle, are (1) on 30 seconds, off 10 minutes; (2) on 25 seconds, off 10 minutes; (3) on 20 seconds, off 10 minutes; and (4) on 15 seconds, off 10 minutes. The water is delivered by a water pipe 41 located toward the upper portion 23 of the housing 20. In a preferred embodiment, the pipe 41 has a spray nozzle 45 located at each end 43. Depending on the size of the bio-reactor 10, there can be more than two spray nozzles 45 or only one spray nozzle 45. The water cycle may be controlled by a selector switch on a control panel (not shown) of the bio-reactor 10.

The agitation cycle is provided by a plurality of spaced-apart and offset mixing composting paddles 70. The mixing paddles 70 are preferably of one-piece construction with the paddle portion 71 being integral to the connecting rod portion 77. The connecting rod portion 77 preferably includes a keyway 79 that receives a complementary shaped key 49 located on the paddle shaft 47. An AC motor 90 is used to rotate the paddle shaft 47, through a chain-driven and gear-reduced arrangement 91, thereby causing the mixing paddles 70 to turn. Standard 110V power is used (compared to prior art composters which required 220V power). A chain tensioner 93A or B ensures that the paddle shaft 47 continues to rotate at the proper speed. The speed and amount of agitation is determined based upon such factors as food-type, processing time, input frequency and total waste. Preferably, the paddles 70 run continuously and at the same speed throughout the agitation cycle and may push or pull their way through the compostable material. The access door 28 to the drum 40 may be equipped with duel inductive-type proximity sensors (not shown) to ensure the door 28 is closed prior to the bio-reactor 10 cycling.

The bio-reactor 10 includes means for preventing the plurality of biochips from entering the outlet water stream. In a preferred embodiment, perforated bottom screens 50 located in the lower portion 21 of the housing 20 and above the base pan 60 of the bio-reactor 10 are used for this purpose. (The base pan 60 is sloped toward the water outlet 33.) The size of the perforations 51 in the screens 50 is very important. If the perforations 51 are too large, then the effluent contains partially decomposed food waste. If the perforations 51 are too small, then decomposed food waste cannot exit the bio-reactor 10, new waste cannot be introduced, and the decomposition process stops. Therefore, the bio-reactor 10 also includes means for limiting the particle size distribution of the decomposed food waste entering the outlet water stream. The perforated bottom screens 50 may be used for this purpose. The bottom screens 50 limit the maximum particle size exiting the bio-reactor 10 to about 0.040″ in diameter.

To prevent the perforations 51 in the bottom screens 50 from becoming blocked by debris, the paddle portion 71 of each mixing paddle 70 includes a wiper blade (or sweeper) 75 at its paddle end 73. The wiper blade 75 may be constructed of polyurethane or its equivalent. Wiper blade 75 may also be constructed of a brush material. The paddle portion 71 is preferably constructed so that its paddle end 73 includes a pair of blades 75 oriented at about a 90° angle to one another, or each blade 75 may be a single piece blade that extends across the normally arranged face surfaces 74 of the paddle end 73 (or across a single, straight face surface 74). The face surface 74 is preferably arranged so that blade 75 is oriented oblique to the direction of travel of the mixing paddle 70. The paddle portion 71 may also be arranged or rotated so that the wiper blade 75 pulls through the compostable material rather than pushes through it.

As each mixing paddle 70 rotates, the paddle 70 aerates the compostable material within the drum and the wiper blade 75 passes over the bottom screen 50 and prevents debris from settling for too long a period of time on the screen 50. Preferably, one wiper blade 75 does not overlap the adjacent wiper blade 75. A spacing of about 1 inch between adjacent wiper blades 75 has proved adequate. Additionally, pipes 53 having a plurality of nozzles 55 may be located below the bottom screens to direct hot water under pressure toward the bottom screens 50. These “screen cleaner” pipes 53 preferably deliver water for 30 seconds and then remain off for 30 minutes. Another pipe 61 located below the bottom screens 50 and opposite the water outlet 33 directs water under pressure toward the discharge water outlet 33. This “pan flush” pipe 61 has a plurality of nozzles 63 and preferably delivers water for 20 seconds and then remains off for 1 hour. (Other screen cleaning and base flush cycles may be used, and the screen cleaner pipes 53 may be eliminated altogether.) The wiper blades 75 in conjunction with the nozzles 55, 63 (or nozzles 63 alone) prevent debris build-up from occurring on the screens and within drum 40.

The control panel (not shown) ma y be fitted with a sensor warning light and dual capacitive liquid level sensors 85 to prevent an overflow condition within the housing 20. The level sensors 85 are located just inside the bio-reactor near the upper portion 23 on the left and right hand sides 25 of the bio-reactor. In the event one or both of these sensors 85 sense an overflow condition for a predetermined amount of time (e.g., 6 seconds), the warning light flashes. If only one sensor 85 indicates the overflow condition, the bio-reactor 10 operates in a normal cycle. However, if both sensors 85 indicate the overflow condition, the bio-reactor 10 will go into safe mode. No fresh water will be added and the motor 90 will go into constant run mode. After a predetermined amount of time passes in safe mode (e.g. 1 hour), the bio-reactor 10 will recheck the sensors 85.

The bio-reactor 10 preferably locates the water inlets 29, 31 and power inputs 35 on a side 25 of the housing 20 so that the back 27 of bio-reactor 10 can go flush up against a wall, thereby saving space. The hot water inlet 31 may be located flush with the base pan 60 of the bio-reactor 10.

The preferred embodiments described above are illustrations which provide enabling examples of a bio-reactor made and practiced according to this invention. The invention itself is defined by the following claims, which cover designs which are equivalent to those illustrated here.

What is claimed is:

1. A bio-reactor for composting food waste and bio-compostable materials, the bio-reactor comprising:
a plurality of mixing paddles, each mixing blade having a paddle shaft and a paddle end, the paddle end including a wiper blade;
each wiper blade being arranged to wipe a portion of a perforated bottom screen.

2. A bio-reactor according to claim 1 further comprising a perforation in the bottom screen being no greater than about 0.040″ in diameter.

3. A bio-reactor according to claim 1 further comprising the wiper blade of at least one mixing paddle in the plurality of mixing paddles being arranged oblique to a direction of travel of the one mixing paddle.

4. A bio-reactor according to claim 1 further comprising a paddle end portion of at least one mixing paddle in the plurality of mixing paddles being a wedge-shaped paddle end.

5. A bio-reactor according to claim 1 further comprising a plurality of nozzles located below the perforated bottom screen and directed to deliver water under pressure toward an outlet end of the bio reactor.

6. A bio reactor according to claim 1 further comprising a plurality of nozzles located below the perforated bottom screen and directed to deliver water under pressure to an underside portion of the perforated bottom screen.

7. A bio reactor according to claim 1 further comprising a first and second set of nozzles located below the perforated bottom screen, the first set of nozzles arranged to deliver hot water under pressure to an underside portion of the perforated bottom screen, the second set of nozzles arranged to deliver hot water under pressure toward an outlet end of the bio reactor.

8. A method for composting bio-compostable materials, the method comprising the steps of:
adding a bio-compostable material to a bio-reactor including a plurality of plastic resin biochips housing a micro-organism; and
repeatedly cycling between a timed water cycle and a timed non-water cycle, the water cycle including a fresh water source
continuously agitating the bio-compostable material during its residence time within the bio-reactor;
the bio-compostable material coming into contact with the plurality of biochips and the water supplied by the water cycle and decomposing and exiting the housing in an outlet water stream.

9. A method according to claim 8 further comprising the step of wiping away bio-compostable material temporarily residing on a bottom screen of the bio-reactor during the continuously agitating step to prevent a lower portion of the bio-reactor from becoming blocked by the bio-compostable material.

10. A method according to claim 9 wherein the wiping step being accomplished by a plurality of mixing paddles each having a wiping blade arranged to contact the bottom screen.

11. A method according to claim 10 wherein the wiping blade is a brush.

12. A method according to claim 10 wherein the wiping blade is arranged oblique relative to a direction of travel of the plurality of mixing blades.

13. A method according to claim 8 further comprising means for preventing the plurality of biochips from entering the outlet water stream.

14. A method according to claim 8 further comprising means for limiting the particle size distribution of the decomposed bio-compostable material entering the outlet water stream.

15. A method according to claim 14 further comprising the maximum particle size in the particle size distribution being about 0.040″ in diameter.

16. A method according to claim 8 further comprising the step of directing water under pressure below a bottom screen and toward a discharge outlet side of the bio-reactor.

17. A method according to claim 8 further comprising the step of directing water under pressure toward an underside of a bottom screen of the bio-reactor.

18. A method according to claim 8 wherein the decomposed food waste exits the housing in the outlet water stream within a range of 24 to 48 hours of the bio-compostable material being added to the bio-reactor.
